(19) United States
(12) Patent Application Publication
(10) Pub. No.: US 2024/0410857 A1
(43) Pub. Date: Dec. 12, 2024

(54) ELECTROPHORETIC MOBILITY SHIFT AS A MOLECULAR BEACON-BASED READOUT FOR MIRNA DETECTION

(71) Applicants: lonita C. GHIRAN, Winchester, MA (US); Beth Israel Deaconess Medical Center, Boston, MA (US)

(72) Inventor: Ionita C. Ghiran, Winchester, MA (US)

(73) Assignee: Beth Israel Deaconess Medical Center, Boston, MA (US)

(21) Appl. No.: 18/274,028

(22) PCT Filed: Jan. 27, 2022
(86) PCT No.: PCT/US2022/014088

§ 371 Date: Jul. 25, 2023

Related U.S. Application Data
(60) Provisional application No. 63/142,348, filed on Jan. 27, 2021.

(51) Int. Cl. G01N 27/447; C12Q 1/68

(52) U.S. Cl. CPC G01N 27/44726; C12Q 1/68; C12Q 2600/178

(57) ABSTRACT

Aspects of the technology described herein relate to systems and techniques for detecting one or more nucleic acids comprising a target sequence of nucleotides, the method comprising incubating molecular beacons (MBs) and a concentration of nucleic acids, each comprising a sequence of nucleotides, wherein the molecular beacons are configured to generate a fluorescence signal when bound with the target sequence, performing electrophoresis by applying voltage, and determining, using the electrophoretic mobility shift of the MBs and nucleic acids during electrophoresis, the presence of the target sequence in the concentration of nucleic acids.

### CROSS-REFERENCE TO RELATED APPLICATIONS

The present application claims the benefit under 35 U.S.C. § 119 (e) of U.S. Patent Application Ser. No. 63/142,348, filed Jan. 27, 2021 under Attorney Docket No. B0662.70113US00, and entitled “ELECTROPHORETIC MOBILITY SHIFT AS A MOLECULAR BEACON-BASED READOUT FOR miRNA DETECTION,” which is hereby incorporated by reference herein in its entirety.

### FEDERALLY SPONSORED RESEARCH

This invention was made with Government support under RO1CA218500, UG3HL147353, and UG3TR002881 awarded by the National Institutes of Health. The Government has certain rights in the invention.

### FIELD

Generally, the aspects of the technology described herein relate to systems and methods of identifying molecules of single-strand DNA (ssDNA) and single-strand RNA (ssRNA), such as a target sequence of nucleotides. Certain aspects relate to detecting the presence of a target sequence in a concentration of nucleic acids using electrophoretic mobility shift of molecular beacons (MBs) and nucleic acids during electrophoresis.

### BACKGROUND

MicroRNA molecules (miRNAs) were first described in the nematode Caenorhabditis elegans in early 1990. These molecules are short, non-coding RNA sequences (19-22 nucleotides (nt)) that primarily function as silencers of RNA expression, and regulators of gene expression. The array of functions of the miRNA-regulated RNA molecules is significant, spanning cell division, growth, differentiation, apoptosis, and migration. The number of confirmed mature miRNAs continues to increase, with their number to date of 1917 precursors, and 2654 mature for Homo sapiens [GRCh38]. More recently, miRNAs have received increased attention for basic biological processes, and as biomarkers in liquid biopsy for disease diagnostics, progression, treatment efficacy and relapse. Quantitative detection of miRNAs in various biological fluids is usually performed using Northern blotting or PCR-based techniques, which are usually laborious and time consuming.

### SUMMARY

According to one aspect of the present application, a method for detecting one or more nucleic acids comprising a target sequence of nucleotides is provided, the method comprising incubating molecular beacons (MBs) with a concentration of nucleic acids, each nucleic acid comprising a sequence of nucleotides, wherein the molecular beacons are configured to generate a fluorescence signal when bound with the target sequence performing electrophoresis by applying voltage and determining, using the electrophoretic mobility shift of the MBs and nucleic acids during electrophoresis, the presence of the target sequence in the concentration of nucleic acids.

In some embodiments, the target sequence corresponds to a mutated sequence.

In some embodiments, the target sequence is a micro ribonucleic acid (miRNA).

In some embodiments, the target sequence is a single strand ribonucleic acid (ssRNA).

In some embodiments, the target sequence is a single strand deoxyribonucleic acid (ssDNA).

In some embodiments, the method further comprises conjugating the concentration of nucleic acids with streptavidin beads.

In some embodiments, the method further comprises obtaining blood of a patient, isolating ribonucleic acid (RNA) from red blood cells (RBCs) of the blood of the patient.

In some embodiments, a gel used in electrophoresis is not stained.

In some embodiments, electrophoresis comprises applying a first voltage for a first period of time and a second voltage for a second period of time.

In some embodiments, the method further comprises determining a measurement indicative of a quantity of nucleic acids comprising the target sequence.

According to one aspect of the present application, a system is provided, the system comprising at least one computer hardware processor, and at least one non-transitory computer-readable storage medium storing processor-executable instructions that, when executed by the at least one computer hardware processor, cause the at least one computer hardware processor to perform a computer implemented method for performing methods described herein. Some aspects of the present application include at least one non-transitory computer-readable storage medium storing processor-executable instructions that, when executed by at least one processor, cause the at least one processor to perform the above aspects and embodiments. Some aspects include an apparatus having a processing device configured to perform the above aspects and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1A shows four exemplary MBs, in accordance with certain embodiments described herein.

FIG. 1B shows fluorometry kinetic assay of the hybridization of the MBs of FIG. 1A and their corresponding target, in accordance with certain embodiments described herein.

FIG. 1C is an exemplary dose dependence analysis of the MBs of FIG. 1A, in accordance with certain embodiments described herein.

FIG. 1D is an exemplary linear regression of MB-miRNAs target analogs hybridization showing the respective r-squared values, in accordance with certain embodiments described herein.

FIG. 2A shows results of gel electrophoresis for MBs hybridized with increasing concentration of hsa-miR-451a target analogs (DNA backbone), in accordance with certain embodiments described herein.

FIG. 2B shows kinetic measurements of the MB-target analog hybridization, measured by gel electrophoresis, in accordance with certain embodiments described herein.

FIG. 2C shows the results of electrophoresis on exemplary MBs incubated with corresponding targets, in accordance with certain embodiments described herein.

FIG. 3A shows the results of gel electrophoresis where MBs were incubated with increasing concentrations of corresponding miRNA analogs (RNA backbone), in accordance with certain embodiments described herein.

FIG. 3B shows the linear regression, calculated using area under the curve (AUC) values measured from positive duplex bands MB-miRNA target analog, in accordance with certain embodiments described herein.

FIG. 3C shows the results of gel electrophoresis where miR451aMB was incubated with same concentration (50 nM) of either a DNA backbone hsa-miR451a or RNA backbone hsa-miR451, in accordance with certain embodiments described herein.

FIG. 4A shows secondary structures of different hybridization patterns between miR451aMB and hsa-miR-451a WT hybridized with WT or mutated sequences, M1, M2, M3, and M4, in accordance with certain embodiments described herein.

FIG. 4B shows a graph representing fluorescence measured by fluorometry, where ten nM of MB were coupled with 500 nm strep streptavidin beads and hybridized with 5 nM of hsa-miR-451a WT analog or mutated sequences (M1 to M4), in accordance with certain embodiments described herein.

FIG. 4C shows a graph representing fluorescence signal measured by flow cytometry displayed as geometric MFI-FITC of MB-streptavidin beads incubated with 5 nM of WT or mutated sequences M1 to M4, in accordance with certain embodiments described herein.

FIG. 4D shows a gel electrophoresis pattern of MB incubated with hsa-miR-451a WT, or mutated sequences M1 to M4, in accordance with certain embodiments described herein.

FIG. 4E shows the area under the curve of MB-WT or MB-Mutated sequences, in accordance with certain embodiments described herein.

FIG. 5A shows the result of electrophoresis for detection of hsa-miR-451a in increasing concentrations of total RNA purified from RBCs isolated from a donor, in accordance with certain embodiments described herein.

FIG. 5B shows the result of electrophoresis using two hundred fifty nanograms of total RNA RBCs isolated from five self-declared healthy donors (labelled D1 to D5) which were incubated with miR451aMB, in accordance with certain embodiments described herein.

FIG. 5C shows a graph of quantitative PCR data obtained from five donors, in accordance with certain embodiments described herein.

FIG. 6A shows representative melting curves calculated for all four MB (e.g., 110, 120, 130, and 140), in accordance with certain embodiments described herein.

FIG. 6B shows flow cytometric kinetic measurements of the MB-miRNAs interaction measured at 0, 1, 5, 10, 20, 30 mins, in accordance with certain embodiments described herein.

FIG. 7A shows results from electrophoresis where MB were incubated with picomolar concentrations of targets (100 to 500 pM) for 30 min at 55° C. followed by gel electrophoresis, in accordance with certain embodiments described herein.

FIG. 7B shows fluorometric results of sub nanomolar concentrations of miRNA targets, in accordance with certain embodiments described herein.

FIG. 7C shows the linear regression calculated using area under the curve measured on positive signal bands, in accordance with certain embodiments described herein.

FIG. 8A shows a dot plot representing gating strategy, in accordance with certain embodiments described herein.

FIG. 8B shows results of electrophoresis used to identify of hybridization between MB and WT-targets\mutation using DNA backbone, in accordance with certain embodiments described herein.

FIG. 9A shows the results of electrophoresis using endogenous miRNA, in accordance with certain embodiments described herein.

FIG. 9B shows the same gel from FIG. 9A after bands were cut, in accordance with certain embodiments described herein.

FIG. 9C shows the results, expressed as Ct of the RT-qPCR analysis of the RNA eluted from the highlighted bands, in accordance with certain embodiments described herein.

FIG. 10 is a table of exemplary MBs and corresponding target sequences, according to some embodiments.

FIG. 11 is a flow diagram showing steps of a method 1100 for detecting one or more nucleic acids comprising a target sequence of nucleotides, in accordance with certain embodiments described herein.

FIG. 12 shows, schematically, an illustrative computer 1200 on which any aspect of the present disclosure may be implemented.

### DETAILED DESCRIPTION

MicroRNAs are short, non-coding RNA sequences (e.g., typically 19-22 nucleotides (nt)) that primarily function as silencers of RNA expression, and regulators of gene expression. The array of functions of the miRNA-regulated RNA molecules is significant, spanning cell division, growth, differentiation, apoptosis, and migration. Quantitative detection of miRNAs in various biological fluids is usually performed using Northern blotting or Polymerase Chain Reaction (PCR)-based techniques. These techniques are usually laborious and time consuming.

To address some of these shortcomings of conventional approaches, the inventors have developed techniques for rapid, and affordable methods for sensitive detection of single-stranded DNA and RNA in point-of-care settings. In particular, the inventors have developed techniques for the use of electrophoretic mobility (e.g., delayed electrophoretic mobility) for detection of target sequences of nucleotides, such as miRNAs, using molecular beacons (MBs).

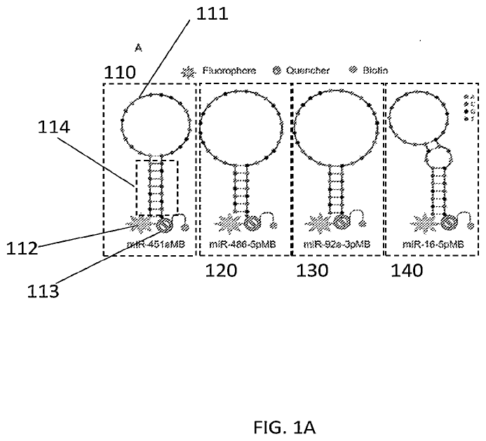

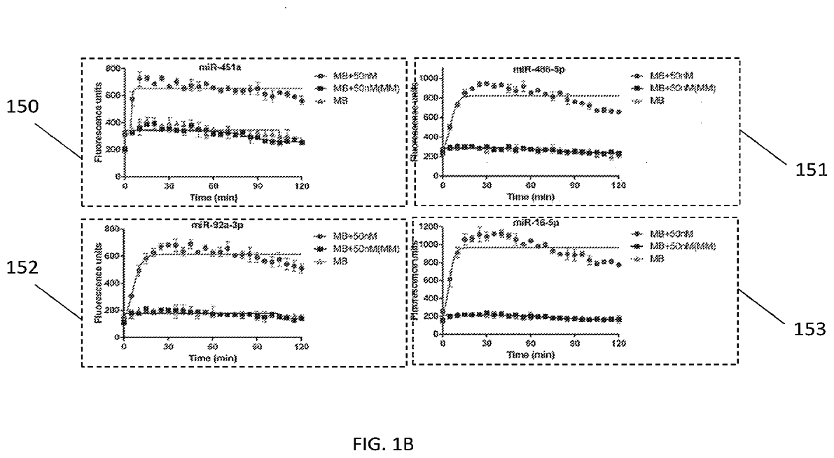

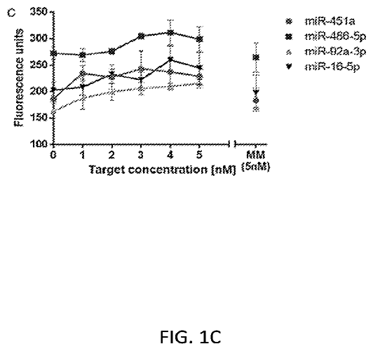

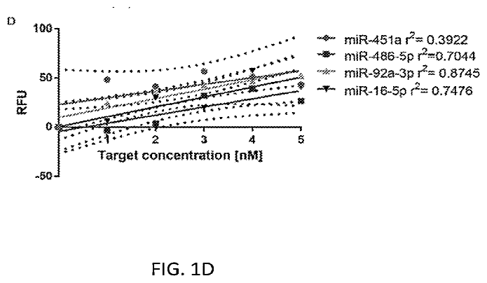

Molecular beacons (MBs) are hairpin-shaped oligonucleotides (RNA or DNA) that contain an anti-sense hybridization sequence matched to a specific target sequence of nucleotides such as single-stranded RNA or DNA molecule. MBs also include a double-stranded stem region, and at its termini, a fluorochrome and a quencher. FIG. 1A shows four exemplary MBs, 110, 120, 130 and 140. In the example of FIG. 1A, the MB 110 has anti-sense hybridization sequence made up of nucleotides 111, fluorophore 112 and quencher 113.

In the absence of the target sequence, the stem sequence keeps the quencher (e.g., 113) and the fluorochrome (e.g., 112) in close proximity preventing the MB from fluorescing. Binding of the MB to the target by the hybridization sequence triggers a conformational change in the stem which opens the beacon and separates the quencher from the fluorochrome, allowing emission of fluorescence upon excitation. Only the binding of the MB with the intended target (e.g., single strand ribonucleic acid (ssRNA) or single strand deoxyribonucleic acid (ssDNA)) should generate a fluorescence signal, although separation of the quencher from the fluorochrome by contaminating ribonucleic acids (RNAses), or temperature-dependent changes in conformation could also have the same results. Using locked nucleic acids (LNA) instead of standard nucleotides when synthesizing the MBs can successfully alleviate this problem. The readouts for the MB-generated signal may involve fluorometry, microscopy, or more recently, when using biotinylated or cell penetrating peptides-conjugated MB, flow cytometry.

More recently, MBs have started to be used successfully not only for the detection of, but also for the differentiation between miRNAs and precursor-miRNAs (pre-miRNA) (the loop sequence) using fluorometry as a readout method. MBs coupled to cell penetrating peptides (CPP) may be used for detection of miRNAs species in both cells and extracellular vesicles using super resolution microscopy and nano flow cytometry. However, the cost of the CPP-MBs and of the necessary microscopes or flow cytometers for detection limits its use in point-of-care settings.

Molecular probes based on DNA self-assembly (referred to herein as DNA nanoswitches) are structures that include, for example, a long ssDNA scaffold (e.g., almost 8000 bps long) that has been titled with complementary oligonucleotides and/or decorated with affinity reagents that can bind to change the topology of the nanoswitch. These changes in topology can be read out using gel electrophoresis due to their effect on electrophoretic mobility. Techniques described herein can be used to demonstrate high-sensitivity, high-specificity detection of protein biomarkers in serum by decorating each nanoswitch with a pair of sandwiching antibodies, in point-of-care (POC) settings. Furthermore, by replacing the antibodies with strands of ssDNA complementary to nucleic acid sequences of interest, this concept has been extended to enable the detection of miRNAs.

Upon hybridization with target sequences (e.g, miRNAs), MBs form a fluorescent duplex with reduced electrophoretic mobility compared to MB alone, thus bypassing the need for additional staining. In addition to emission of light, the location of the fluorescent band on the gel acts as an orthogonal validation of the target identity, further conforming the specificity of binding. According to some embodiments, the limit of detection of this approach is between 10 to 110 pM, depending on the MB sequence. The method may be sensitive enough to detect specific red blood cell miRNAs molecules in total RNA, with single nucleotide specificity, in less than 30 minutes.

One aspect of the present application includes a method for detecting one or more nucleic acids comprising a target sequence of nucleotides, the method comprising incubating molecular beacons (MBs) with a concentration of nucleic acids, each nucleic acid comprising a sequence of nucleotides, wherein the molecular beacons are configured to generate a fluorescence signal when bound with the target sequence, performing electrophoresis by applying voltage, and determining, using the electrophoretic mobility shift of the MBs and nucleic acids during electrophoresis, the presence of the target sequence in the concentration of nucleic acids.

In one aspect of the present application, an electrophoretic-based method is provided which identifies the detection of specific ssRNA and ssDNA molecules, for example, by the coincident output of both delayed electrophoretic mobility and emission of fluorescence. The technology developed by the inventors allows for an approach that does not require any staining, as the signal is provided by the fluorescence of the beacon following the binding to the target ssRNA or ssDNA. Moreover, as the two means of detection are orthogonal (fluorescence and changes in the electrophoretic speed of the single MB compared to the MB-target complex), this adds an additional level of specificity to the method. As no washing or amplification steps are required, it can be used as a sensitive and easy-to-use assay in a laboratory setting or at the point-of-care.

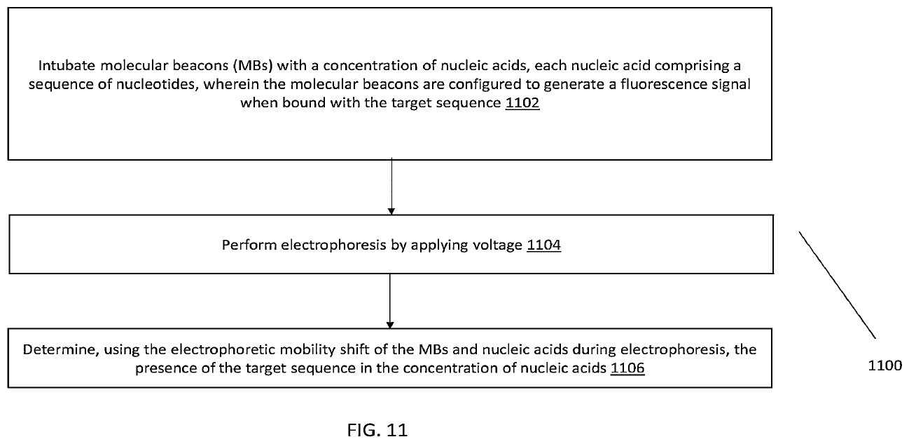

FIG. 11 is a flow diagram showing steps of a method 1100 for detecting one or more nucleic acids comprising a target sequence of nucleotides, in accordance with certain embodiments described herein.

In step 1102, the method includes incubating molecular beacons (MBs) with a concentration of nucleic acids, each nucleic acid comprising a sequence of nucleotides, wherein the molecular beacons are configured to generate a fluorescence signal when bound with the target sequence.

In step 1104, the method includes performing electrophoresis by applying voltage. For example, according to some embodiments, a constant voltage may be applied for a predetermined amount of time. In some embodiments, a first constant voltage may be applied for a first amount of time, and subsequently, a second constant voltage may be applied for a second amount of time, for example, as described herein.

In step 1106, the method 1100 includes determining, using the electrophoretic mobility shift of the MBs and nucleic acids during electrophoresis, the presence of the target sequence in the concentration of nucleic acids. According to some examples, the method may also include determining the presence of the target sequence in the concentration of nucleic acids using a coincident output of both the electrophoretic mobility and emission of fluorescence. According to some examples, the method may also include quantifying an amount of the target sequence in the concentration of nucleic acids using a coincident output of both the electrophoretic mobility and emission of fluorescence.

miRNA-MB Hybridization Detection by Fluorometry

According to some embodiments, the MBs may be diluted prior to incubation. For example, MBs may be diluted in 100 μL of dPBSIX to a final concentration of 50 nM, and then incubated with different concentrations of synthetic miRNA oligonucleotide target analog (from 0 to 50 nM) in 96 well plates (e.g, such as Corning™ 96-Well clear bottom, black walls) for 30 min at 37° C. or 55° C. The fluorescence intensity of each well may then be measured (λEx 495 nm; λEm 521 nm) by a microplate reader (e.g., Synergy HT Multi-Mode, Biotek, Winooski, VT, USA). For the kinetics assay, MBs were diluted in 100 μL of dPBS1X to a final concentration of 50 nM, and then incubated with either 0, 50 nM target analog or 50 nM mismatch sequences in 96 well plates. Fluorescence (λEx 495 nm; λEm 521 nm) was acquired at 55° C. every 5 min using a BioTek Synergy 4 fluorometer.

### Coupling MB to Streptavidin Beads

Prior to conjugation, MBs were diluted to 50 nM in dPBSand heated at 90° C. for 5 minutes, as per manufacturer instructions. Streptavidin beads were diluted in dPBS to a concentration of 10,000 beads/uL. MBs were added to 500 nm streptavidin beads and incubated at 37° C. for 15 min. Once the incubation was completed, the MB-conjugated beads were washed 3 times in 1 mL of dPBS and centrifuged at 5600 xg for 5 min to remove any free MBs. After washing, MB-beads were resuspended to a final volume of 200 uL with a final concentration of 50 nM of synthetic miRNA oligonucleotide target analog. Using flow cytometry, miRNA-MB hybridization was analyzed over a time course (0, 1, 5, 10, 20, and 30 min of incubation at 37° C.), and the efficiency of miRNA-MB hybridization was also determined for different miR-451a mismatch analog sequences (WT, and mutations 1, 2, 3, and 4, see Table 1). Target and mutation analog sequences were incubated at 37° C. for 30 min before analysis.

miRNA-MB Hybridization Detection by Flow Cytometry

As the size of the streptavidin beads was 500 nm, the CytoFLEX LX flow cytometer was set up in the “nanoparticle detection mode” as previously reported. Briefly, within the violet pod, the 450/45 bandpass was placed in position one and the 405/10 bandpass was placed in position two (Detector One). VSSC was used as the trigger parameter, and VSSCA linear versus SSCA log was plotted for bead population determination. The settings were optimized using Polysciences NIST Nanoparticle bead mix with sizes ranging from 80-500 nm, and set as follows: SSC: 58 V, VSSC: 50 V, FITC: 95 V, the FITC channel was used to measure the fluorescein fluorescence of the bead-attached MBs. For consistency, 15,000 events in the 500 nm gate population were recorded for each specimen. Each sample was acquired at a rate of approximately 10,000 events per second.

miRNA-MB Hybridization Detection by Gel Electrophoresis

Twenty microliters of 50 nM MB were incubated with various concentrations of synthetic miRNAs or DNA oligonucleotide target analogs for 30 min, mixed with Gel Loading dye (6X), and then loaded into a Novex TBE 4-20% gels. Gel electrophoresis was performed with constant voltage for 10 min at 100V, and then the voltage was increased to 150V for an additional 40 min. The MB fluorescence signal was visualized using 6-Fluorescein or Alexa 488 channel on a ChemiDoc MP Imaging System (Bio-rad, Hercules, CA). Exposure times were set on “Manual”, and varied depending on the sample between 10 to 300 seconds. The gel electrophoresis kinetic assay was performed by incubating 10 nM MB with 10 nM of synthetic DNA oligonucleotide target analogat various time points (15 seconds, 1, 5, 10, 20, and 30 min). The samples were prepared in a final volume of 20 uL using a 96-well plate, and kept at 370 C. As all the time points had to be run simultaneously, DNA oligonucleotide target analog was added in a staggered order starting with the 30 min time point. After 10 minutes, the target was added to the 20 min time point well, and so forth. One-minute before the 30-minute incubation time expired, the 20 uL in each well were mixed with 4 uL of Gel Loading dye and loaded into the gel. For the “0 min” time point the beacon was mixed with the DNA oligonucleotide target analog, gel Loading dye, and then added directly into the gel. Once loaded, the samples were run at 95V, constant voltage, for 1.5 hours. The gel was imaged as described above.

### Blood Draw, and RBC Isolation

The current study was approved by the Beth Israel Deaconess Medical Center Institutional Review Board (IRB). Four milliliters of fresh whole blood were obtained via venipuncture using 5 mL of Vacutainer EDTA tubes (BD, Franklin Lakes, NJ) from 5 self-declared healthy volunteers. First, plasma was separated from whole blood by centrifugation of 500 xg for 10 min. RBCs were isolated by diluting the whole blood 1:1 with HBSS——, and passing the blood through an Acrodisc white blood cells (WBC) removal syringe filter (Pall Corporation, NY). RBCs were then washed in 1 mL of HBSS—— three times at 500 xg for 10 min each, to remove any residual platelets.

RNA Isolation, cDNA Synthesis and qPCR

According to some embodiments, the concentration of nucleic acids may include RNA from a patient. In some examples described herein, red blood cells (RBCs) were collected from 5 self-declared healthy donors as described above. RBC small RNA was purified (e.g., using miRNeasy Mini Kit (Qiagen) following manufacturer's protocol). Isolated RNA was quantified using Qubit™ microRNA Assay Kit in a Qubit 4 Fluorometer (Thermo Fisher). Gel bands were cut using a scalpel, and RNA was eluted from gel using MinElute Gel extraction kit (Qiagen). Complementary DNA (cDNA) synthesis was performed using TaqMan Advanced miRNA cDNA Synthesis Kit (Thermo Fisher). Quantitative PCR (qPCR) was performed using TaqMan Fast Advanced Master Mix (Thermo Fisher) in triplicates with the primers hsa-miR451a (Taq Man assay ID 001105), hsa-miR486-5p (TaqMan assay ID 478128_mir), hsa-miR-92a-3p (assay ID, 000431), and hsa-miR16-5p (assay ID, 000391) in a 7500 Fast Real-time PCR System (Applied Biosystems, US.). The qPCR thermal cycling conditions were set as follow: Step 1: Enzyme activation at 95° C. for 20 sec, 1 cycle; Step 2: Denaturing at 95° C. for 3 sec, and anneal/extend at 60° C. for 30 sec, 40 cycles. Analyses of the data (Ct values for each replicate) were performed using the standard curve method, and the threshold baseline was adjusted to 1.7 for all samples.

### Statistical Analysis of the Data

Linear regression and R-squared values (r²) were used to test linearity between increasing concentrations of MB-target and detected fluorescence. Limit of detection (LOD) was calculated based on area under the curve (AUC) values obtained from 0 nM (background) and 0.1 nM from three independent experiments. AUC from gel bands was measured using ImageJ software (National Institutes of Health, Bethesda, Maryland).

FIGS. 1A-1D show exemplary fluorometry-based quantification of MB-miRNAs target analog hybridization.

As described herein, FIG. 1A shows four exemplary MBs, 110, 120, 130 and 140. In particular, FIG. 1A shows the conformation of the four MBs including MB 110 miR451aMB, MB 120 486-5pMB, MB 130 92a-3pMB, and MB 140 16-5pMB at 55° C. In the example of FIG. 1A, the MB 110 has anti-sense hybridization sequence made up of nucleotides 111, fluorophore 112 and quencher 113. Each of the four MBs of FIG. 1A is composed of a 5′-end 6-fluorescein (FAM), a stem sequence (e.g., 114), a loop sequence (shown above the loop sequence) complementary to a corresponding target sequence, a 3′-end internal quencher, linker and a biotin molecule.

As described herein, in the absence of the target sequence, the stem sequence keeps the quencher (e.g., 113) and the fluorochrome (e.g., 112) in close proximity preventing the MB from fluorescing. Binding of the MB to the target by the hybridization sequence triggers a conformational change in the stem which opens the beacon and separates the quencher from the fluorochrome, allowing emission of fluorescence upon excitation. Only the binding of the MB with the intended target (e.g., single strand ribonucleic acid (ssRNA) or single strand deoxyribonucleic acid (ssDNA)).

FIG. 1B shows fluorometry kinetic assay of the hybridization of the MBs of FIG. 1A and their corresponding targets (e.g., miR451aMB-target analog hybridization). For example, graph 151 shows the kinetic assay of MB 110, graph 152 shows the kinetic assay of MB 120, graph 153 shows the kinetic assay of MB 130, and graph 154 shows the kinetic assay of MB 140. The fluorescence in each of the graphs 151-154 reach a peak between 15 and 30 min of incubation at 55° C.

FIG. 1C is an exemplary dose dependence analysis of the MBs 110, 120, 130 and 140 from 1 to 5 nM, for 30 min at 55° C. FIG. 1D is an exemplary linear regression of MB-miRNAs target analogs hybridization showing the respective r-squared values.

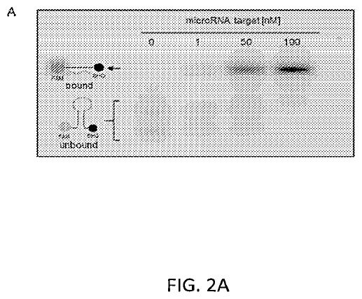

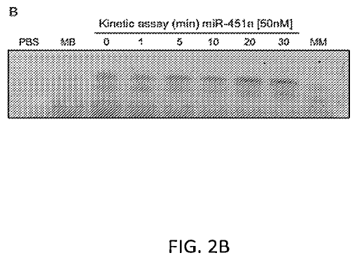

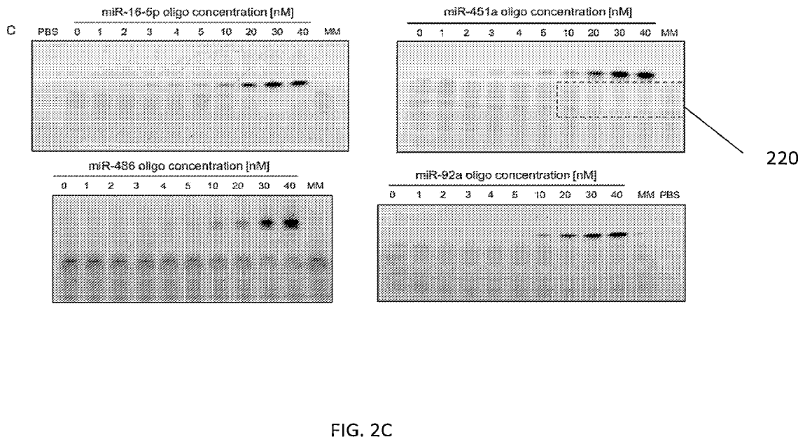

FIGS. 2A-C show an example of gel electrophoresis used to detect MB-miRNA target analog (DNA backbone) hybridization, using, for example, the method 1100 of FIG. 11.

In the example of FIG. 2A, results of gel electrophoresis for MBs hybridized with increasing concentration of hsa-miR-451a target analogs (DNA backbone) can be shown. The top band represents the duplex MB-Target hybridization (lower electrophoretic mobility) while bottom bands represents unbound MB (higher electrophoretic mobility).

In FIG. 2B, kinetic measurements of the MB-target analog hybridization are shown, measured by gel electrophoresis. The band representative of the duplex MB-target analog hybridization reached a maximum fluorescence intensity after 20 min of incubation.

FIG. 2C shows the results of electrophoresis on exemplary MBs incubated with corresponding targets. In FIG. 2C, the same electrophoretic mobility pattern seen for miR451a MB-miRNA target analog hybridization was seen for miR486-5pMB, miR92a-3pMB, and miR16-5pMB incubated with their respective miRNA target analogs. Highlighted in box 220 is the depletion of unbound MB fluorescence due to MB-target analog hybridization. MM represents a mismatch target (not complementary sequence).

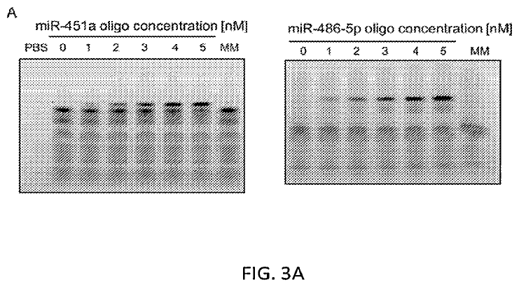

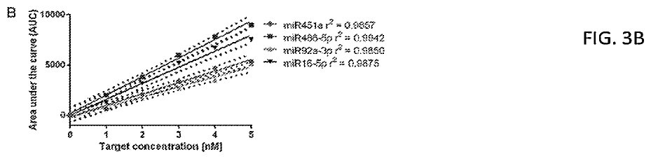

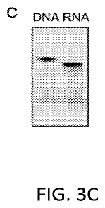

In FIG. 3A-C, it can be seen that DNA and RNA backbones miRNA analogs have different electrophoretic mobility patterns.

FIG. 3A shows the results of gel electrophoresis where MBs were incubated with increasing concentrations of corresponding miRNA analogs (RNA backbone). The pattern of top (duplex MB-target analog), and bottom (unbound MB) bands seen when using DNA backbone for miRNA analog was maintained, although RNA backbone miRNA analogs produced a positive MB-target analog band closer to unbound MB.

FIG. 3B shows the linear regression, calculated using area under the curve (AUC) values measured from positive duplex bands MB-miRNA target analog.

FIG. 3C shows the results of gel electrophoresis where miR451aMB was incubated with same concentration (50 nM) of either a DNA backbone hsa-miR451a or RNA backbone hsa-miR451.

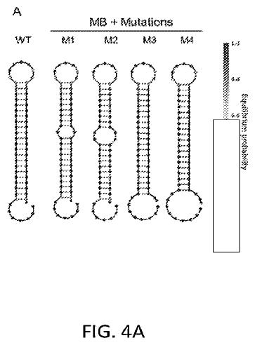

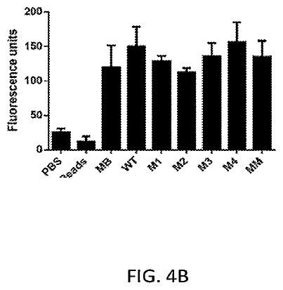

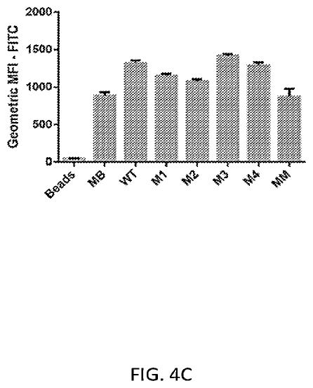

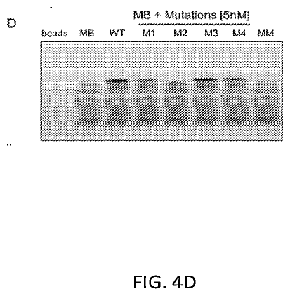

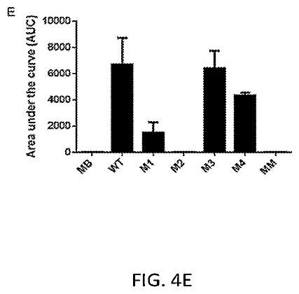

FIG. 4A-E shows an example of using electrophoretic mobility shift to differentiate certain hsa-miR-451a mutation sequences.

FIG. 4A shows secondary structures of different hybridization patterns between miR451aMB and hsa-miR-451a WT hybridized with WT or mutated sequences, M1, M2, M3, and M4. The bases are shaded to represent a heat map representing the minimum free energy between the hybridization of each base pair.

FIG. 4B shows a graph representing fluorescence measured by fluorometry, where ten nM of MB were coupled with 500 nm strep streptavidin beads and hybridized with 5 nM of hsa-miR-451a WT analog or mutated sequences (M1 to M4).

FIG. 4C shows a graph representing fluorescence signal measured by flow cytometry displayed as geometric MFI-FITC of MB-streptavidin beads incubated with 5 nM of WT or mutated sequences M1 to M4.

FIG. 4D shows a gel electrophoresis pattern of MB incubated with hsa-miR-451a WT, or mutated sequences M1 to M4.

FIG. 4E shows the area under the curve of MB-WT or MB-Mutated sequences.

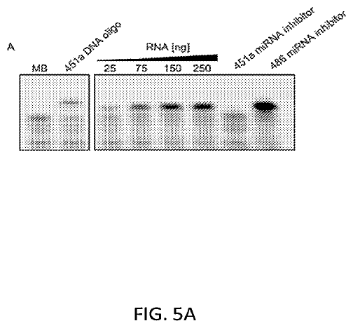

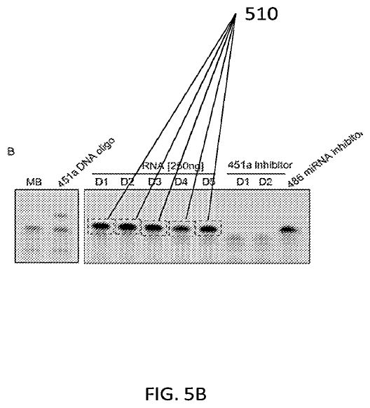

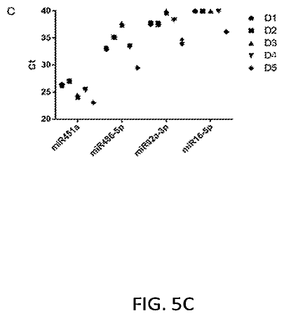

FIG. 5A-C shows an example of identification of endogenous hsa-miR-451a by MB hybridization and electrophoretic mobility shift.

FIG. 5A shows the result of electrophoresis for detection of hsa-miR-451a in increasing concentrations of total RNA purified from RBCs isolated from a donor. The miR451a signal did not form when the isolated total RNA was preincubated with miR451a inhibitor.

In the example of FIG. 5B, two hundred fifty nanograms of total RNA RBCs isolated from five self-declared healthy donors (labelled D1 to D5) were incubated with miR451aMB. The MB-miRNAs hybrid bands (labelled rectangles 510) were cut, and the eluted RNA was prepared for qPCR.

In the example of FIG. 5C, quantitative PCR data obtained from five donors shows the lowest Ct values for miR451a, followed by miR-16-5p. miR92a was not identified in any of the samples, and the sample were represented by Ct of 40. The graph confirms the identity of the target in MB-miRNAs bands.

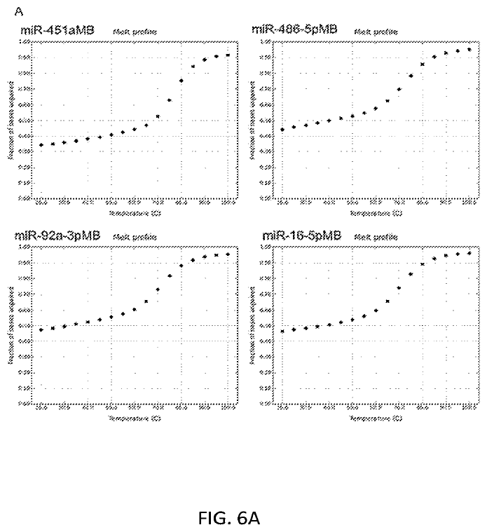

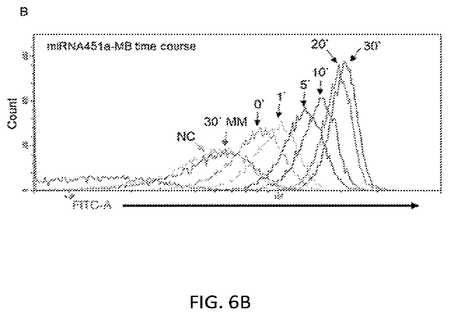

FIGS. 6A-B show characterization of melting curves from MB.

FIG. 6A shows representative melting curves calculated for all four MB (e.g., 110, 120, 130, and 140) calculated, for example, using Nupack software.

FIG. 6B shows flow cytometric kinetic measurements of the MB-miRNAs interaction measured at 0, 1, 5, 10, 20, 30 mins.

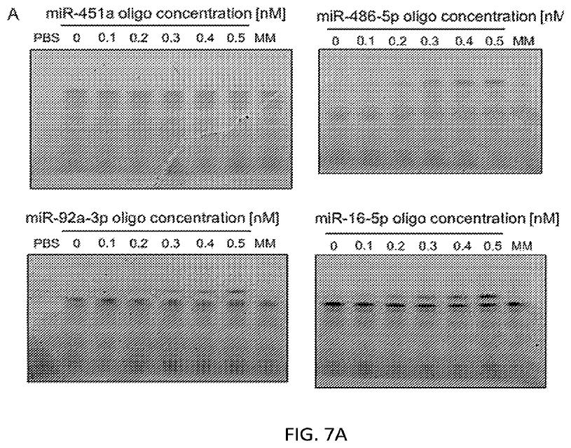

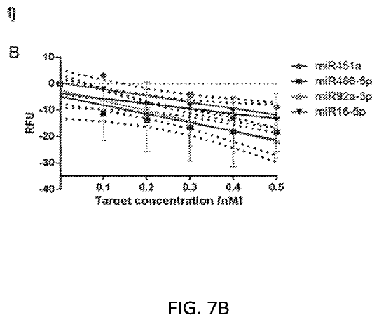

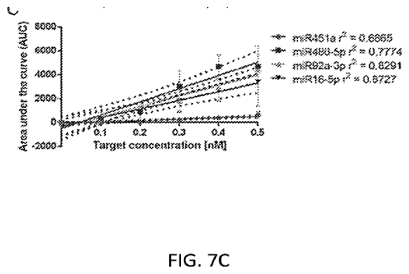

FIG. 7A-C show picomolar detection of MB-target hybridization using electrophoretic mobility shift.

In the example of FIG. 7A, MB were incubated with picomolar concentrations of targets (100 to 500 pM) for 30 min at 55° C. followed by gel electrophoresis.

FIG. 7B shows fluorometric results of sub nanomolar concentrations of miRNA targets.

FIG. 7C shows the linear regression calculated using area under the curve measured on positive signal bands. The experiments of FIG. 7 were performed three times with similar results.

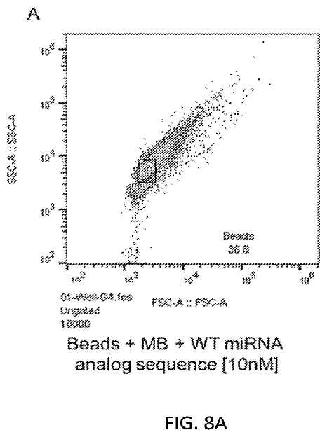

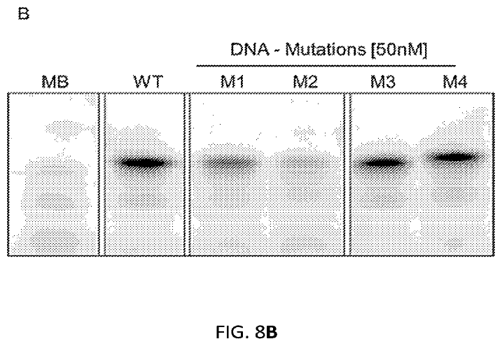

FIGS. 8A-B show detection of single and double mutation targets using MB.

In the example of FIG. 8A, MB were coupled with 500 nm streptavidin beads, and incubated with 10 nM of WT or mutations sequences (M1 to M4) for 30 min at 55° C. Flow cytometry was used to detect fluorescence in the beads population (within the gate). FIG. 8A shows a dot plot representing gating strategy.

FIG. 8B shows results of electrophoresis used to identify of hybridization between MB and WT-targets\mutation using DNA backbone. The pattern found was similar to that of RNA backbone with decrease in fluorescence in mutation 1, 2, and 4, compared to the WT. Three independent experiments were performed.

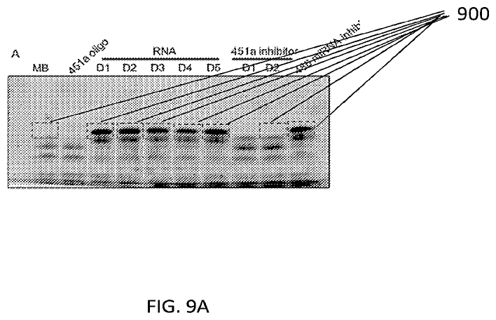

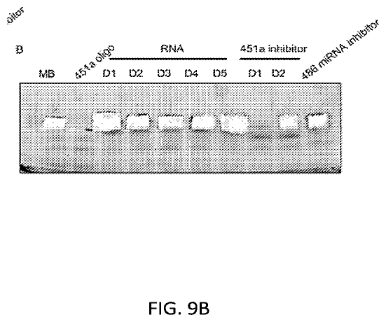

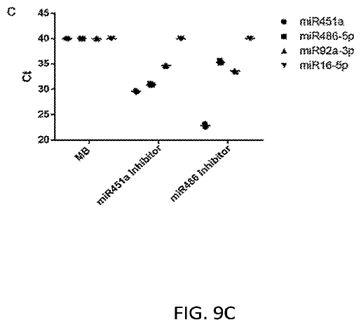

FIGS. 9A-C show identification of endogenous miRNA by MB hybridization and electrophoretic mobility shift.

FIG. 9A shows the results of electrophoresis using endogenous miRNA. In FIG. 9A, the regions 900 shows where MB-miRNAs bands were cut.

FIG. 9B shows the same gel from FIG. 9A after bands were cut.

FIG. 9C shows the results, expressed as Ct of the RT-qPCR analysis of the RNA eluted from the highlighted bands.

### Materials and Methods

Below is a description of exemplary materials that may be used in the processes described herein.

### Reagents

Dulbecco's phosphate-buffered saline (dPBS, 2.6 mMKCl, 1.47 mM KH2 PO4, 137 mMNaCl, and 8.05 mM Na2HPO4), Hanks' Balanced Salt Solution (HBSS——, no calcium, no magnesium), Invitrogen Novex TBE Running Buffer (5X), and Novex TBE Gels, 4-20% were obtained from Thermo Fisher Scientific (Waltham, MA). Gel Loading Dye, Purple (6X), no SDS was obtained from New England Biolabs (Ipswich, Massachusetts). Five hundred nanometer Streptavidin beads were purchased from Bangs Laboratories (Fishers, IN). MiRCURY LNA miRNA Inhibitors (antimiRs) were obtained from Qiagen (Germantown, MD).

### Molecular Beacons and Targets Sequences

Molecular beacons and synthetic miRNAs or DNA oligonucleotide target analogs were obtained from Integrated DNA technologies IDT (Coralville, IA). All MBs were conjugated with a 5′ end 6-carboxyfluorescein (λEx 495 nm; λEm 517 nm), and at the 3′ end an internal ZEN quencher, followed by an 18-atom hexa-ethyleneglycol spacer (ISp18), and a biotin. The mutated miRNAs had the following modifications: M1, mutation from C to A in the 10th position; M2, mutation from CC to AA in the 10th and 11th positions; M3, mutation from U to C in the 22nd position (M3); and M4, mutation from UU to CC in the 21stand 22ndposition. All the MBs and corresponding target sequences used for this project are shown in Table 1.

### Results

The example of FIGS. 1A-D, 2A-C, 3A-C, 4A-E, 5A-C, 6A-B, 7A-C, 8A-B, and 9A-C show designs and tests for four MBs (miR451aMB, miR486-5pMB, miR92a-3pMB, and miR16-5pMB) to detect mature human miRNAs enriched in red blood cells (RBCs), and plasma (hsa-miR451a, hsa-miR486-5p, hsa-miR92a-3p, and hsa-miR-16-5p). The MBs hairpin conformations are shown in FIG. 1A. At 55° C., the MB show different loop structures, due to their unique target miRNAs complementary sequence. In silico melting curve analysis showed similar MB loops dissociation. Subsequently, the fluorescence is measured over time (e.g., every 5 min for 2 h) where the fluorescence generated by the hybridization between MBs and their respective miRNAs target analog (50 nM, FIG. 1B). The fluorescence peak was achieved between 15 to 30 min for all four MBs tested. Negative controls consisting of MB without target (MB, FIG. 1B), or mismatched miRNAs target analog (MB+50 nM (MM), FIG. 1B) showed minimal increase in fluorescence during the experiment. Different hybridization efficiencies were detected among the tested MBs. The fluorescence background values were higher for miR451aMB (360-380 fluorescence units (FU)) with 2-fold increase over background. The other species, miR486-5pMB, miR92a-3pMB, and miR16-5pMB showed approximately 200 FU as a background, and 4.5, 3.5, and 5.6 fold increase over background, respectively. The values of the mismatched miRNAs target analog control (not complementary) showed similar values as the MB alone control. The effect of background fluorescence on the detection sensitivity becomes more apparent at low concentrations of the miRNAs target analogs, from 1 to 5 nM (FIG. 1C), where the linearity of the signal over the background range (r2) drops to 0.874 for miR92a-3pMB and to 0.392 for miR451aMB (FIG. 1D). The low r² value for miR451aMB is not surprising as the background fluorescence of this MB had the highest value of the MB tested. The high background fluorescence seen in the MBs sample alone, could be explained by the incomplete quenching of the fluorochrome, the presence of free dye in the MB preparations, or a combination of both. To test for these possibilities, MBs were coupled to streptavidin beads, and after washing the beads, the miRNAs target analogs were added and the fluorescence was measured over time (5 seconds, 1, 2, 5, 10, 20, and 30 min) by flow cytometry. The results from flow cytometry suggest that the high MB background fluorescence was due not by the presence of free dye in MB solution but mostly from incomplete quenching or MB dissociation.

The experiments represented in FIGS. 1A-D, 2A-C, 3A-C, 4A-E, 5A-C, 6A-B, 7A-C, 8A-B, and 9A-C show that hybridization of the molecular beacon to the target alters its electrophoretic properties.

The principle of MB-based nucleic acid detection using gel electrophoresis, relies on: i) a different electrophoretic speed on the MB-target complex vs. miRNA/MB alone, and ii) the binding-dependent fluorescence of the MB (FIG. 2A). The sensitivity of the method was tested by incubating 100 nM of miR451aMB with increasing concentrations of miRNAs target analogs (DNA backbone 1, 10, and 100 nM). Gel electrophoresis was perform as described in methods section. The fluorescent band intensity representative of the MB-target hybridization (higher band, arrow) increased, as expected in a dose-dependent manner. Simultaneously, the gel also showed a progressive decrease in the fluorescence pattern of the unbound MB (lower bands), showing the depletion of the MBs paralleling the increase of the target (FIG. 2A). Then a kinetic experiment was performed, by incubating 50 nM MBs for increasing amounts of time, 15 sec, 1, 5, 10, 20, or 30 min, with 50 nM of the target analog at 37° C. FIG. 2B shows that the signal is visible even when the co-incubation time is approximately 15 seconds, reaching a fluorescence peak in 20 minutes, after which the signal plateaus. These results are consistent with the fluorometry and flow cytometry data. Next, the ability of the MBs to detect low concentrations of miRNA target analogs was tested. Using DNA backbone as a miRNA target analog, a positive signal could be detected using 2 nM of targets for miR451aMB, miR486-5pMB and miR16-5pMB, and 10 nM for miR92a-3pMB (FIG. 2C). As expected, the gradual depletion of free MBs can be seen as the concentration of miR-451a target analog increases (FIG. 2C). This process is similar to the depletion of the nucleotide pool near the front in the lanes with positive PCR product. The rest of the fluorescence signal seen in the lanes below the free MBs bands is likely free dye or fragments of MBs.

According to the examples of FIGS. 1A-D, 2A-C, 3A-C, 4A-E, 5A-C, 6A-B, 7A-C, 8A-B, and 9A-C show that DNA and RNA targets analogs generate similar electrophoretic patterns with different sensitivities.

As the initial targets used for these experiments were synthesized using a DNA backbone, and the goal was to validate the approach using miRNAs obtained from biologically relevant samples, testing to determine whether the signals obtained from RNA and DNA targets analogs were comparable was performed. The experiments reported in FIG. 2 were repeated, using miRNAs synthesized with RNA backbone. The results show that the sensitivity of detection increased when the target analog was RNA-based, reaching the 1 nM range for all MB tested (FIG. 3A). When area under the curve (AUC) for the positive gel bands, was measured, all MB showed a strong linearity with a r2 above 98% (FIG. 3B). As the signal in RNA target analogs was stronger than that obtained with DNA, subnanomolar concentrations of the target analogs (100 to 500 pM) were tested. A positive band with 100 pM was detected for all RNA-backbone miRNA target analogs except for miR451aMB, which generated a detectable band when using 200 pM. Due to the lower signal-to-noise ratio when imaging these gels, the r2 values obtained from AUC dropped to 0.6-0.8. Based on the measurement of the AUC from three different gels using subnanomolar concentration (100 to 500 pM), the limit of detection (LOD) was calculated for all four MB, as follows: miR451aMB, LOD of 40 pM; miR486-5pMB, LOD of 110 pM; miR92a-3pMB, LOD of 50 pM, and miR 16-5pMB, LOD of 10 pM. A visual inspection of the DNA and RNA gels show that the RNA-backbone targets seemed to migrate closer to the unquenched MBs upper band compared to the DNA-target. This was verified in observation by incubating, in adjacent lanes, miR451aMB with targets synthesized using either DNA or RNA backbone. The results (FIG. 3C) showed that the relative electrophoretic mobility of the MB hybridization with miRNA using RNA backbone was indeed faster than the corresponding DNA analog. This difference is likely due to the presence of an additional hydroxyl residue on C2 of ribose compared to deoxyribose, thus increasing the overall negative charge of the RNA molecule compared to that of the corresponding DNA sequences.

In the example shown in FIGS. 1A-D, 2A-C, 3A-C, 4A-E, 5A-C, 6A-B, 7A-C, 8A-B, and 9A-C, electrophoretic mobility can identify hybridization of MB to mutated miRNA target analogs.

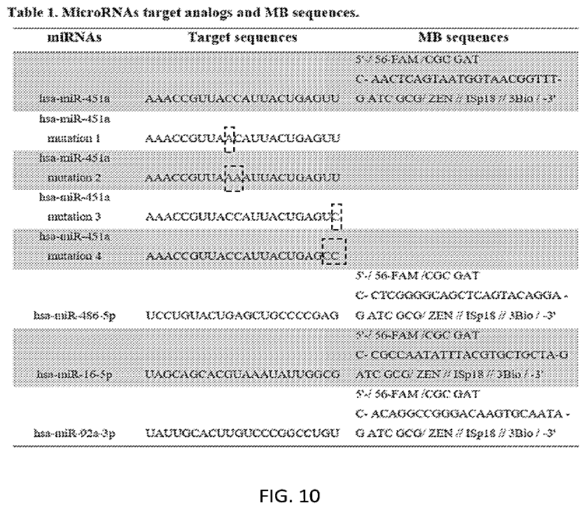

Current molecular beacon-based methods for detection of point mutation afford identification of single mutation as long as the mismatched nucleotide is flanked on either side by at least one functional base pair. The ability of the electrophoretic mobility shift to differentiate between the wild type miRNA target analog and several 1 and 2 nt mutated miRNA target analog sequences was tested (FIG. 10; Table 1). Analyses of the MB hybridization with WT or mutated sequences performed byNupack software (available at http://www.nupack.org/) indicated the presence of a mismatched loop in the middle of the duplex MB-target analog sequence when MB was incubated with mutated sequences 1 (M1), and 2 (M2) (FIG. 4A). The ability of fluorometry, flow cytometry and electrophoretic mobility shift to detect differences in the hybridization of the MB-WT, or MB-Mutated sequences was compared. For this comparison, miR451aMB was conjugated with 500 nm streptavidin beads, and were incubated with either 5 nM of WT or mutated miR451a analog sequences (RNA backbone, M1 to M4). The results show that while fluorometry analysis was unable to detected any significant signal from either WT or mutated targets over control (FIG. 4B), flow cytometry identified accurately the targets with 1 and 2 mutations located in the middle of the sequence, but failed to identify the end mutations at the end of the sequences (FIG. 4C). The electrophoresis-based method correctly identified M1 (center, C to A) and M2 (center, CC to AA) mutations, and while it failed to distinguish mutation M3 (5′-end, U to C), rendered a slightly slower and less bright band (higher on the gel) for mutation M4 (5′-end, AA to CC). When the target analogs were synthesized using a DNA backbone, the electrophoretic pattern was similar to that observed when using the RNA-backbone target, however, the 5′-end (AA to CC) generated a duplex band slightly dimmer but with significantly lower electrophoretic mobility than that of the WT. Area under the curve profiles of the MB-WT or MB-mutated sequences hybridization bands is shown in FIG. 4E.

In the examples shown in FIGS. 1A-D, 2A-C, 3A-C, 4A-E, 5A-C, 6A-B, 7A-C, 8A-B, and 9A-C it can be seen that electrophoretic mobility shift can identify endogenous miRNA species.

As all the results presented above were obtained using synthetic DNA or RNA backbones as miRNA analogs, it was next sought to validate the approach using purified RNA from blood cells, specifically RBCs. Total RNA from RBCs was isolated from 5 self-declared healthy donors and the levels of hsa-miR-451a were measured using both RT-qPCR and gel electrophoresis. For the electrophoretic mobility assay, 100 nM of miR451aMB with increasing amounts (25, 75, 150, and 250 ng) of total RBC RNA were incubated. As a positive control, hsa-miR-451a, and as negative hsa-miR-486-5p inhibitor were used. Similar to the results obtained using synthesized RBC miRNAs, the fluorescence intensity of the miR451aMB-target band increased with the amount of RBC RNA added to the reaction. The positive band did not form when the RNA was pre-incubated with a specific hsa-miR-451a miRCury LNA Inhibitors (anti-miRs). The MB-miR451a hybridization was not affected when incubating the RNA with a hsa-miR-486-5p inhibitor, further confirming the identity of the positive band as the presence of a duplex MB-hsa-miR-451a (FIG. 5A, top). To further confirm the identity of the MB-miRNAs duplex, two fluorescent negative bands (control samples) were cut, one in the miR451aMB lane, and a second one in the miR451aMB-hsa-miR-451a Inhibitors, as well as the fluorescently positive bands obtained from the hybridization of miR451aMB (FIG. 5B, highlighted in red). The gel fragments were then eluted, the RNA was isolated, and qPCR was performed to detect four highly present miRNAs in RBC: hsa-miR451a, 486-5p, 92a-3p, and 16-5p. Quantitative PCR results indicate that areas with positive fluorescence bands corresponding to the putative miR451aMB-RNA target complex showed the lowest Ct values for hsa-miR-451a, as compared to the others miRNAs from all five donors (FIG. 5C). None of the miRNAs were detected in the negative control bands (miR451aMB alone). Furthermore, the Ct values from hsa-miR-451a increased from 22 to 29 when incubating the RNA previously with hsa-miR-451a inhibitor.

### Discussion

For several decades, fluorometry was the standard method used to quantify the fluorescence triggered by the binding of MB to their target sequences. This method affords, unlike cell-based approaches, a tight control over experimental conditions such as MB and target concentration, buffer pH and composition, ion content, as well as changes in temperature during experiments. Furthermore, this method allows MB fluorophore multiplexing, conjugation of MB with gold nanoparticles or qDots. In addition to the stable and controlled conditions, the frequency of sample interrogation can be set anywhere between seconds to tens of minutes. A drawback of fluorometry is bulk reading of the reaction solution when the presence of free fluorophore, incomplete quenching, and degraded beacon will significantly increase the noise, and decrease the sensitivity.

Methods described herein include a gel electrophoresis-based readout method to detect specific miRNAs in the picomolar range. The built-in on/off fluorescence reporter generates light only when the MB is hybridized with the intended target, circumventing the need for additional staining steps, and due to the delayed electrophoretic mobility of the MB-target duplex, the location of the positive fluorescent band also acts as an orthogonal confirmation of the specificity of target binding.

The gel electrophoresis-based readout is fully applicable to identifying various ssRNA and ssDNA molecules found in biological fluids, such as, viruses, circulating RNA complexes, cell-free DNA, and nucleic acids associated with extracellular vesicles. As certain sequences of interest may not be readily available for MB hybridization due to either secondary structure, or the presence of interacting proteins, incubating the sample with helper oligos, which flank the target site, may also improve the chances for a positive MB signal. For double-stranded nucleic acids, using the direct MB approach as described here is not feasible, unless the selected region is present in a loop of the molecule where the beacon has access, or when using a CRISPR/cas9-MB tandem approach, as was recently reported in living cells.

The sensitivity limit of this approach depends primarily on the brightness of the fluorochromes, the sensitivity of the imaging device, and the autofluorescence of agarose gels. Using quantum dots (qDots) as MB fluorochromes has been used successfully for both, increasing the sensitivity of the signal, and affording longer integration times with limited photobleaching. As the size of qDots is between 2-6 nm this approach should not hinder the migration of the MB or MB-target complex on the gel. For transcripts longer than miRNAs, using several MB and FRET MB tandems would also lower the detection limit and provide an opportunity for multiplexing, as well as testing for insertions/deletions/mutations in given sequences. However, longer RNA molecules, as is common in mRNA molecules or certain viruses, may require mechanical sheering or enzymatic cleavage prior to gel detection to allow effective gel penetration of the genetic material.

During gel electrophoresis, the relative mobility of migrating molecules depends on their size, conformation, and when running the samples in SDS-free conditions, their overall charge. It was found (FIG. 3C) that the difference in the relative electrophoretic mobility between MB and DNA or RNA backbone target analogs was in fact significant, likely due to the presence of an additional hydroxyl residue on C2 of ribose compared to deoxyribose. This appeared to effectively increase the overall negative charge of RNA molecules, as well as the relative mobility of the duplex MB-RNA complex compared to the MB-DNA complex.

When the ability of gel electrophoresis to identify several mutations in the miRNA sequence was investigated (FIG. 4), we found that the location and number of the mismatched nucleotides in the electrophoretic pattern of the duplex miR451aMB-WT or miR451aMB-mutated sequences was critical for understanding the gel profile. The data is consistent with previous reports showing only partial loss in fluorescence signal for single nucleotide mutation mismatch in target sequences. Obtaining a binary answer regarding the presence of a point mutation in the target sequence flanked by non-mutated nucleotides, requires a more elaborate approach involving the measurement of thermal denaturation profiles of the MB-targets, followed by identification of the gap between the transition temperatures of matched and point-mutated duplexes. Once the two temperatures are known, the readout is performed within the temperature range where the matched duplex is still fluorescent, and the single nucleotide mismatched target is no longer hybridized to the MB. results in FIG. 3C and Supplemental FIG. 4C, show that even when the target sequence has 2 terminal mutations, the electrophoretic profile is able to differentiate the mutated sequences from those of the WT sequence, especially when the target has a DNA backbone.

As integrated systems for gel electrophoresis and visualization become more sensitive and affordable, the method introduced here could be an effective means for fast, specific, and sensitive identification of a variety of nucleic acid targets in point-of-care friendly settings.

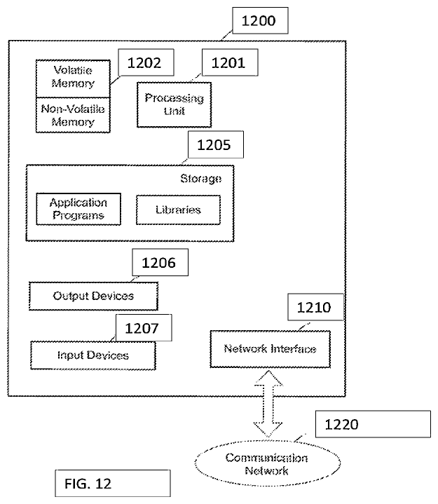

FIG. 12 shows, schematically, an illustrative computer 1200 on which any aspect of the present disclosure may be implemented, including, for example, the process 1100. In the example shown in FIG. 12, the computer 1200 includes a processing unit 1201 having one or more processors and a computer-readable storage medium 1202 that may include, for example, volatile and/or non-volatile memory. The memory 1202 may store one or more instructions to program the processing unit 1201 to perform any of the functions described herein. The computer 1200 may also include other types of computer-readable medium, such as storage 1205 (e.g., one or more disk drives) in addition to the system memory 1202. The storage 1205 may store one or more application programs and/or resources used by application programs (e.g., software libraries), which may be loaded into the memory 1202.

The computer 1200 may have one or more input devices and/or output devices, such as output devices 1206 and input devices 1207 illustrated in FIG. 12. These devices may be used, for instance, to present a user interface. Examples of output devices that may be used to provide a user interface include printers, display screens, and other devices for visual output, speakers and other devices for audible output, braille displays and other devices for haptic output, etc. Examples of input devices that may be used for a user interface include keyboards, pointing devices (e.g., mice, touch pads, and digitizing tablets), microphones, etc. For instance, the input devices 1207 may include a microphone for capturing audio signals, and the output devices 1206 may include a display screen for visually rendering, and/or a speaker for audibly rendering, recognized text.

In the example of FIG. 12, the computer 1200 may also include one or more network interfaces (e.g., network interface 1210) to enable communication via various networks (e.g., communication network 1220). Examples of networks include local area networks (e.g., an enterprise network), wide area networks (e.g., the Internet), etc. Such networks may be based on any suitable technology, and may operate according to any suitable protocol. For instance, such networks may include wireless networks and/or wired networks (e.g., fiber optic networks).

### [Standard Language Follows]

While the above description has described various circuitry and methods for operating such circuitry in the context of ultrasound devices, the circuitry and methods may be used in the context of other electronic devices as well.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within +20% of a target value in some embodiments, within +10% of a target value in some embodiments, within +5% of a target value in some embodiments, and yet within +2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

1. A computer-implemented method for detecting one or more nucleic acids comprising a target sequence of nucleotides, the method comprising:
incubating molecular beacons (MBs) with a concentration of nucleic acids, each nucleic acid comprising a sequence of nucleotides, wherein the molecular beacons are configured to generate a fluorescence signal when bound with the target sequence;
performing electrophoresis by applying voltage; and
determining, using the electrophoretic mobility shift of the MBs and nucleic acids during electrophoresis, the presence of the target sequence in the concentration of nucleic acids.

2. The computer-implemented method of claim 1, wherein the target sequence corresponds to a mutated sequence.

3. The computer-implemented method of claim 1, wherein the target sequence is a micro ribonucleic acid (miRNA).

4. The computer-implemented method of claim 1, wherein the target sequence is a single strand ribonucleic acid (ssRNA).

5. The computer-implemented method of claim 1, wherein the target sequence is a single strand deoxyribonucleic acid (ssDNA).

6. The computer-implemented method of claim 1, further comprising conjugating the concentration of nucleic acids with streptavidin beads.

7. The computer-implemented method of claim 1, further comprising:
obtaining blood of a patient;
isolating ribonucleic acid (RNA) from red blood cells (RBCs) of the blood of the patient.

8. The computer-implemented method of claim 1, wherein a gel used in electrophoresis is not stained.

9. The computer-implemented method of claim 1, where electrophoresis comprises applying a first voltage for a first period of time and a second voltage for a second period of time.

10. The computer-implemented method of claim 1, wherein the method further comprises determining a measurement indicative of a quantity of nucleic acids comprising the target sequence.

11. A system, comprising:
at least one computer hardware processor; and
at least one non-transitory computer-readable storage medium storing processor-executable instructions that, when executed by the at least one computer hardware processor, cause the at least one computer hardware processor to perform a computer implemented method for detecting one or more nucleic acids comprising a target sequence of nucleotides, the method comprising:
incubating molecular beacons (MBs) with a concentration of nucleic acids, each nucleic acid comprising a sequence of nucleotides, wherein the molecular beacons are configured to generate a fluorescence signal when bound with the target sequence;
performing electrophoresis by applying voltage; and
determining, using the electrophoretic mobility shift of the MBs and nucleic acids during electrophoresis, the presence of the target sequence in the concentration of nucleic acids.

12. The system of claim 11, wherein the target sequence corresponds to a mutated sequence.

13. The system of claim 11, wherein the target sequence is a micro ribonucleic acid (miRNA).

14. The system of claim 11, wherein the target sequence is a single strand ribonucleic acid (ssRNA).

15. The system of claim 11, wherein the target sequence is a single strand deoxyribonucleic acid (ssDNA).

16. The system of claim 11, further comprising conjugating the concentration of nucleic acids with streptavidin beads.

17. The system of claim 11, further comprising:
obtaining blood of a patient;
isolating ribonucleic acid (RNA) from red blood cells (RBCs) of the blood of the patient.

18. The system of claim 11, wherein a gel used in electrophoresis is not stained.

19. The system of claim 11, where electrophoresis comprises applying a first voltage for a first period of time and a second voltage for a second period of time.

20. The system of claim 11, wherein the method further comprises determining a measurement indicative of a quantity of nucleic acids comprising the target sequence.


SEQUENCE LISTING (16 sequences)

SEQ ID NO: 1 - RNA, 22 nt, Artificial Sequence
  Synthetic
aaaccguuaccauuacugaguu

SEQ ID NO: 2 - RNA, 22 nt, Artificial Sequence
  Synthetic
aaaccguuaacauuacugaguu

SEQ ID NO: 3 - RNA, 22 nt, Artificial Sequence
  Synthetic
aaaccguuaaaauuacugaguu

SEQ ID NO: 4 - RNA, 22 nt, Artificial Sequence
  Synthetic
aaaccguuaccauuacugaguc

SEQ ID NO: 5 - RNA, 22 nt, Artificial Sequence
  Synthetic
aaaccguuaccauuacugagcc

SEQ ID NO: 6 - RNA, 22 nt, Artificial Sequence
  Synthetic
uccuguacugagcugccccgag

SEQ ID NO: 7 - RNA, 22 nt, Artificial Sequence
  Synthetic
uagcagcacguaaauauuggcg

SEQ ID NO: 8 - RNA, 22 nt, Artificial Sequence
  Synthetic
uauugcacuugucccggccugu

SEQ ID NO: 9 - DNA, 23 bp, Artificial Sequence
  Synthetic
caactcagtaatggtaacggttt

SEQ ID NO: 10 - DNA, 23 bp, Artificial Sequence
  Synthetic
cctcggggcagctcagtacagga

SEQ ID NO: 11 - DNA, 24 bp, Artificial Sequence
  Synthetic
ccgccaatatttacgtgctgctag

SEQ ID NO: 12 - DNA, 23 bp, Artificial Sequence
  Synthetic
cacaggccgggacaagtgcaata

SEQ ID NO: 13 - DNA, 36 bp, Artificial Sequence
  Synthetic
cgcgatcaactcagtaatggtaacggtttgatcgcg

SEQ ID NO: 14 - DNA, 36 bp, Artificial Sequence
  Synthetic
cgcgatcctcggggcagctcagtacaggagatcgcg

SEQ ID NO: 15 - DNA, 36 bp, Artificial Sequence
  Synthetic
cgcgatcacaggccgggacaagtgcaatagatcgcg

SEQ ID NO: 16 - DNA, 36 bp, Artificial Sequence
  Synthetic
cgcgatccgccaatatttacgtgctgctagatcgcg